(19) United States
(12) Patent Application Publication
(10) Pub. No.: US 2010/0146660 A1
(43) Pub. Date: Jun. 10, 2010

(54) PROCESS FOR MICROPROPAGATION OF POGOSTEMON CABLIN FROM A MERISTEMATIC EXPLANT

(75) Inventors: Sreenivasachar Murali Krishnapuram, Maharashtra (IN); Neera Pandey, Maharashtra (IN); Shailendra Baviskar, Maharashtra (IN); Ghanshyam Maurya, Maharashtra (IN)

Correspondence Address: MINTZ, LEVIN, COHN, FERRIS, GLOVSKY AND POPEO, P.C, One Financial Center, BOSTON, MA, 02111, US

(73) Assignee: RELIANCE LIFE SCIENCES PVT. LTD., Navi Mumbai, Maharashtra (IN)

(21) Appl. No.: 12/295,034

(22) PCT Filed: Mar. 30, 2007
(86) PCT No.: PCT/IN2007/000133

§ 371 Date: Feb. 22, 2010

(30) Foreign Application Priority Data
Mar. 31, 2006 (IN) 487/MUM/2006

(51) Int. Cl. A01H 4/00

(52) U.S. Cl. 800/268

(57) ABSTRACT

The present invention provides a commercially viable process for in vitro propagation of patchouli (Pogostemon cablin). The process involves direct organogenesis from meristematic explants employing a simple and cost effective media and shows about 90% survival upon transfer to in vivo conditions.

### CROSS REFERENCE TO RELATED APPLICATIONS

This application claims the benefit of provisional Indian Application number 487/MUM/2006 filed on Mar. 31, 2006, which is hereby entirely incorporated by reference.

### FIELD OF THE INVENTION

The present invention relates to an efficient method for in vitro micropropagation of Pogostemon cablin (patchouli) to generate healthy true-to-type plants. The methods of the present invention employ meristematic explants and provide media and culture conditions which produce a large number of patchouli plants.

### BACKGROUND INFORMATION

Patchouli (i Pogostemon cablin), belonging to family Lamiaceae, is the source of patchouli oil, which is widely used to give a solid foundation and lasting character to a fragrance. The decoction from the leaves is used in Chinese medicines.

Patchouli (Pogostemon cablin) is a member of the mint family. The patchouli plant, or Pogostemon cablin, is an upright, bushy, evergreen perennial herb with lightly fragrant leaves, and white, violet-marked flowers. Native to tropical Asian countries, patchouli is widely cultivated all over the tropics and subtropics including Indonesia, the Philippines, Malaysia, India, southern China, Seychelles, and Brazil.

Patchouli oil is distilled from fermented leaves. Other Pogostemon species as well as similar species produce inferior oils. The best quality oil is produced from plant materials harvested near plantations where there is less chance of damage to plant materials prior to processing. Patchouli oil scent has staying power and is perceptible for weeks or months. At high concentrations, patchouli scent can be sickeningly sweet, but a strong aroma is a sign of superior quality.

Patchouli has a long history in southern Asia and the Far East as incense, body and garment perfume, and insect and leech repellent. Ink in China and India was once perfumed with patchouli.

Indeed, patchouli oil is used in the flavoring industry and is to be found as an ingredient in toiletries, cosmetics, breath fresheners, incense, insecticides, disinfectants, and commercial food flavoring. Patchouli oil is used in perfumery and this oil mixes well with many essential oils including vetiver, sandalwood, frankincense, bergamot, cedarwood, myrrh, jasmine, rose and the citrus oils. Patchouli is employed as a fragrance component in cosmetic preparations, soaps, and hair removal creams because of its masking effect on noxious odors. Patchouli oil is used in temples as incense. Patchouli essential oil is used as a topical remedy for skin problems such as acne, eczema, inflamed, cracked, chapped and irritated skin. It is known as a cell rejuvenator and helpful in healing wounds and scars. As an antifungal, Patchouli oil has been used to treat athlete's foot. For the hair, patchouli oil has been used for dandruff and to aid oily hair.

In the East, patchouli oil has been used to prevent spread of infection due to its antifungal and antibacterial properties. In traditional Chinese medicine, patchouli is used in combination with other herbs to provide relief for colds and flu, fever and chills, headache, nausea, vomiting, diarrhea, abdominal pain, malarial and dysenteric disease, and bad breath. In aromatherapy, patchouli oil is utilized as a relaxant for nervous exhaustion, depression, stress-related complaints, and low libido. Patchouli is not a seasonal crop and can provide regular income throughout the year seven months after planting.

Essential oils are highly concentrated substances extracted from various parts of aromatic plants and trees. These oils are the result of the secondary metabolism of the plant and they form the very basis of the flavor and fragrance industry. Aromatic plants and oils have been used for thousands of years dating back to ancient civilizations to heal, enhance, soothe and excite the body and spirit.

Therefore, in view of the above, there is a need to provide methods for micropropagation of patchouli plants which are economical and allow product of true-to-type, disease-free plants.

### Plant Tissue Culture

Micropropagation is the in vitro regeneration of plants from organs, tissues, cells or protoplast using techniques such as tissue culture for developing true-to type resultant plants of a selected genotype. In general, tissue from a plant commonly known as an explant is isolated from a plant whose multiplication is desired to create a sterile tissue culture of that species in vitro. From explants, a culture is initiated. Once a culture is stabilized and growing well in vitro, multiplication of the tissue or regeneration of entire plant can be carried out. Shoots (tips, nodes or internodes) and leaf pieces are commonly used but cultures can be generated from many different tissues. Juvenile tissues generally respond best. Besides the source of the explants, the chemical composition of the culture medium and the physical environment of cultures have been found to be of a great influence on the regeneration capacity, multiplication ratio, and growth and development of new plants in the culture system. Therefore, one needs to optimize these factors for individual plant species.

Several attempts have been made to regenerate patchouli plants from leaf and node callus. However, tissue regeneration through a callus stage is vulnerable to somaclonal variations and hence will not ensure true-to-type plants from mother plants. In addition, all of the above studies used non-meristem tissue, which is more likely to be infected with disease than meristem tissue. So there is a need for micropropagation methods that produce a large number of true-to type plants of patchouli.

### OBJECTS OF THE INVENTION

The principal aim of the present invention is to develop a commercially viable process for in vitro mass culture of Patchouli.

It is still an object of this present invention to provide a simple and faster process, for production of the true to type plants of elite variety.

It is still an object of this invention to develop a process suitable for commercial production of disease free high yielding plants of uniform quality.

It is also an object of the present invention to identify explants, media and culture conditions for producing maximum regenerates of Patchouli plant by in vitro mass culture.

It is an important aspect of the present invention to provide an optimized process for surface sterilization of explants without damaging the isolated tissues.

In other aspect of the present invention there is provided the best suitable nutrient media supplemented with optimum growth regulators and other components required for different modes and phases of regeneration. Only one media is used for initiation, multiplication and elongation.

It is an important aspect of the present invention to provide the optimum growth conditions with respect to physical parameters like temperature, relative humidity, photoperiod and light intensity for all the stages of culture.

It is an important aspect of the present invention to provide the optimum sub-culture interval during in vitro culture.

It is also an additional aspect of the present invention to provide the hardening protocol for the regenerated plants with as much as about 100% rate of survival in the field.

### SUMMARY OF THE INVENTION

The present invention provides the use of meristem as explants for direct organogenesis giving rise to true-to-type plants.

In one aspect the invention provides methods for producing a true-to-type clone of a Pogostemon cablin mother plant by selecting a mother plant; isolating a meristematic explant from the plant; culturing the meristematic explant in initiation medium to generate shoots; culturing the shoots in proliferation and elongation medium to generate elongated shoots, where the proliferation and elongation medium contains benzyl adenine (BA) and adenine sulfate (AS); culturing the elongated shoots in rooting medium to generate plantlets, where the rooting medium contains indole acetic acid (IAA); and culturing the plantlets to product a true-to-type clone of the Pogostemon cablin mother plant. In particularly preferred embodiments, the initiation medium, proliferation and elongation medium, and rooting medium have the same concentration of basal salts.

In certain embodiments, the meristematic explant is treated to reduce microbial contamination.

In preferred embodiments, the meristematic explant is from a shoot tip or a nodal bud, most preferably, a shoot tip. In particularly preferred embodiments, the shoot tip contains either apical or axillary bud tissue.

In preferred embodiments, the BA is at a concentration from about 0.5 mg/L to about 2.0 mg/L; preferably, about 0.5 mg/L, about 1.0 mg/L, about 1.5 mg/L, or about 2.00 mg/L; and most preferably at a concentration of about 0.5 mg/L.

In other preferred embodiments, the AS is at a concentration from about 1 mg/L to about 10 mg/L; preferably, at about 1.0 mg/L, about 5 mg/L, or about 10 mg/L; most preferably, at a concentration of about 5 mg/L.

In other preferred embodiments, the IAA is at a concentration from about 1 mg/L to about 10 mg/L; preferably, at a concentration from about 1.0 mg/L, about 5 mg/L, or about 10mg/L; and most preferably, at a concentration of about 5 mg/L.

The inventors of the present invention have developed a commercially viable process for in vitro mass culture of patchouli by successfully identifying explants and culture conditions that produce true-to-type patchouli plants.

### BRIEF DESCRIPTION OF THE FIGURES

The following drawings form the part of the present invention and are included to substantiate and demonstrate the important aspects of the disclosure. The present invention may be better understood by the following drawings in combination with the detailed description of the specific embodiments presented herein.

FIG. 1 shows a mother plant.

FIG. 2 shows shoots with roots.

FIG. 3 shows plants for primary hardening.

FIG. 4 shows plants in the greenhouse for hardening.

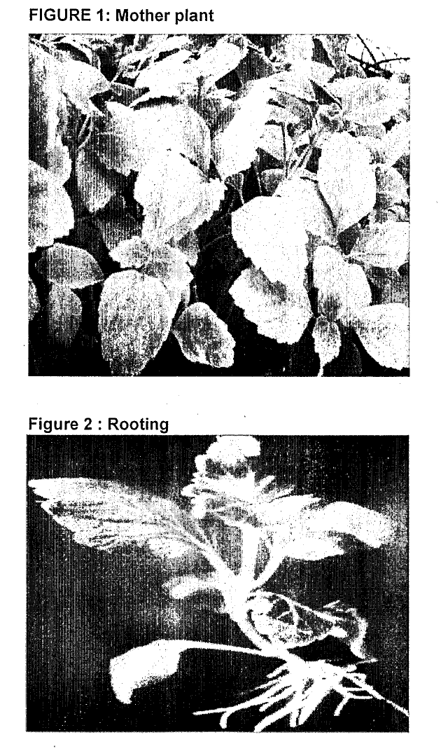

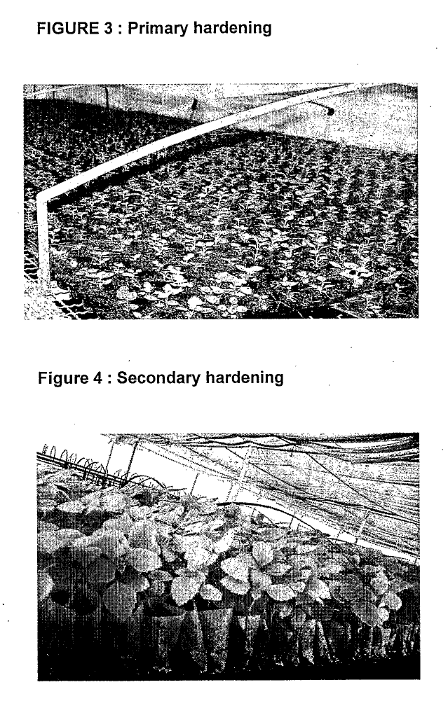

### DETAILED DESCRIPTION OF THE INVENTION

### Definitions

“Micropropagation” refers to the in vitro regeneration of plants from organs, tissues, cells or protoplasts and the true-to-type propagation of a selected genotype using in vitro culture technique.

“Callus” refers to an unorganized or undifferentiated mass of proliferative cells produced either in culture or in nature.

“True-to type propagation” means that all characteristics present in mother plant will also be present in next generation, i.e., the plantlets will be the true type of the mother plant.

“Fungicide” means any chemical substance that destroys and inhibits the growth of fungi.

“Insecticide” means any substance, synthetic or organic, which inhibits, kills, or destroys insects.

“MS” refers to Murashige and Skoog's medium.

“IBA” refers to Indole-3-butyric acid.

“FYM” refers to farm yard manure which can be like compost.

“M-45” refers to Dithane M-45.

“BAP” refers to 6-benzyl amino purine.

“BA” refers to benzyl adenine.

The present invention provides a commercially viable process for in vitro mass culture of patchouli for large-scale multiplication of true-to-type plants.

### General

The present invention provides a process which offers many advantages over the prior art such as the use of meristematic explants.

The present invention provides a method for propagation of true-to-type Pogostemon cablin which uses meristematic explants and cultures them in proliferation and elongation medium containing benzyl adenine (BA) and adenine sulfate (AS) and rooting media containing indole acetic acid (IAA). In contrast to the existing methods, the method produces plants which do not pass through the callus phase and therefore have reduced or nonexistent somaclonal variations with a high success rate. In particularly preferred embodiments, the initiation medium, proliferation and elongation medium, and rooting medium have the same concentration of basal salts.

In some embodiments, the process has steps including, but not limited to, selecting the healthy mother plants, preparing the mother plant, isolating the explant from a mother plant, cleaning the explants, sterilizing the explants by primary and secondary sterilization, inoculating the explants on culture initiation medium, transferring the cultures to proliferation and elongation medium, transferring the elongated shoots to rooting medium, subjecting in vitro grown plantlets to primary and secondary hardening, and transferring of the hardened plantlets to fields.

### Selection and Preparation of the Mother Plants

In certain embodiments, the mother plant are elite plants. The most elite plants are selected by assessing the plants for size, weight, general growth, appearance and absence of infection or contamination.

The mother plants may be treated to either maintain a disease-free state or to treat an existing disease. Decontamination can be performed by spraying the plants with agents such as fungicides, insecticides, pesticides or the like. Preferred fungicides for the pretreatment of the mother plant include, but are not limited to, Bavistin™, Captan™, Dithane™, Thiram™, Thiovit™ or the like, and or combinations thereof at a concentration of about 0.05% to 2%. Preferred insecticides for the pretreatment of the mother plant include, but are not limited to, Roger™, Nuvacron™, Fastac™, Ultracid™, 40-WP, Thiodane™, at a concentration of about 0.005% to 0.02%.

### Explants

The present invention provides a method for efficient in vitro mass culture of Pogosteon cablin using explants from meristematic tissue. It is the meristematic tissue of a plant which carries all the genetic information of mother plants. Moreover, meristematic cells do not have the alterations in their DNA sequence which are found in differentiated cells or tissues. Therefore the plants regenerated using meristematic tissue as an explant are true-to-type to the mother plants.

In preferred embodiments, the meristem tissue is from shoot tip or nodal buds. In preferred embodiments, the shoot trip has apical or axillary buds. In the most preferred embodiments the contemplated explant is shoot tip with apical bud.

Explants used in the present invention are preferably selected from healthy, fresh disease-free plants. In preferred embodiments the explant may be isolated from mother plants growing in various locations, both wild and cultivated.

### Preparation of Explants for Culture

### Cleaning of Explants

In some embodiments the explants are cut from the healthy mother plant and subjected to cleaning and surface sterilization treatment prior to inoculation in culture media.

For example, in certain embodiments apical and axillary buds are cut and cleaned with mild detergent like 0.5-5% Tween-20 Solution with intermittent shaking for 30-60 minutes washing thoroughly with demineralised water.

### Sterilization of Explants

In other embodiments, the explants are sterilized prior to inoculation on the media. Various agents are employed such as mild detergent, fungicide, surface sterilizing agent, or the like and or combinations thereof. The explants may be subjected to single or multiple rounds of sterilization.

For example, the explant may go through a primary sterilization step with the fungicide Bavistin and then go through a secondary sterilization with a surface sterilization agent like sodium hypochlorite or mercuric chloride.

In some embodiments, the cleaned explant is subject to primary sterilization by treating the explants with a solution containing a fungicide like Bavistin 0.05% to 2% for 30-60 minutes, rinsing with sterile water, and cutting the nodal segments to about 3-7 cm. The cleaned explants are then subject to secondary sterilization in a laminar flow bench by treating with a surface sterilizing agent like mercuric chloride 0.01 to 0.2% for 2 to 5 minutes and rinsing with sterile distilled water thrice.

### Culture of Explants

The present invention provides a method for efficient in vitro mass culture of Pogostemon cablin using meristematic explants and in preferred embodiments culturing the explants in initiation, proliferation and rooting medium having the same concentration of basal salts, where the proliferation and elongation medium contains benzyl adenine (BA) and adenine sulfate (AS) and the rooting media contains indole acetic acid (IAA). The composition of the initiation media can vary and will depend on the particular combination and concentration of hormones in the other culture media.

Micropropagation typically involves the following steps: 1) culturing explants in initiation media to generate multiple shoots, 2) transferring shoots to proliferation and elongation media, 3) transferring the elongated shoots to rooting media, 4) hardening the plantlets, and 5) transferring the hardened plantlets to fields.

The initiation, proliferation and rooting medium can be selected from Murashige & Skoog, Gamborg's, Vacin & Went, White's, Schenk & Hildebrandt or the like.

Basal media can also be supplemented with various carbon sources. The carbon source may be sucrose or glucose, typically, at a concentration of about 2-5%. The carbon source may be also be a sugar alcohol like myo-inositol, typically, at a concentration of about 50-500 mg per liter.

In some embodiments the basal media will include gelling agents such as agar, alginic acid, carrageenan, gellan gum. Typical concentrations are 0.5-1%.

In preferred embodiments of the present invention the culture medium for initiation, proliferation and elongation, and rooting is Murashige & Skoog medium with sucrose and solidified with agar.

### Phytohormones in Media

In the preferred embodiments, the proliferation and elongation media contains benzyl adenine (BA) and adenine sulfate (AS) and the rooting media contains indole acetic acid (IAA).

Depending upon the type of phytohormone used, the amount used thereof will vary. The phytohormone may be incorporated at a concentration from about 0.01 mg per liter to about 10 mg per liter. Preferably, the BA is at a concentration from about 0.5 mg/L to 2 mg/L, preferably at about 0.5 mg/L, 1.0 mg/L, 1.5 mg/L, or 2 mg/L; the AS at a concentration from about 1.0 mg/L to about 10 mg/L, preferably at about 1.0 mg/L, 5 mg/L, or 10 mg/L; and the IAA at a concentration from about 1 mg/L to about 10 mg/L, preferably at about 1 mg/L, 5 mg/L, 7 mg/L, or 10 mg/L.

### Culture Conditions

In certain embodiments, the culture conditions (i.e., light cycle, light intensity, media, temperature, relative humidity) are the same throughout the initiation, proliferation and elongation, and rooting stages. Subculturing is performed as necessary; preferably, every 3-4 weeks.

### EXAMPLE

The following steps are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Step 1: Selection of Healthy Mother Plants

The shoots of healthy elite mother plant of patchouli from field were collected. The mature mother plants were from a farm in Gujarat, India. The mother plants were assessed for their size, weight and general growth, appearance, and absence of infection.

### Step 2: Treating the Mother Plants

The mother plants were decontaminated by spraying with Bavistin at a concentration of about 1%.

Step 3: Isolating Explants from Elite Variety of Mother Plant

The apical and the axillary buds from the elite mother plants were isolated and washed thoroughly under running water to remove the dirt adhered to it. Approximately 2-8 cm nodal cuttings with inherent axillary buds and apical shoots were used as explants.

### Step 4: Cleaning of the Explant

Approximately 5 cm long pieces of the nodal segments were cut and cleaned with 2% Tween-20 solution and cleaned with a brush for about 30 minutes and then thoroughly washed with demineralized water.

### Step 5: Sterilisation of the Explant

The cleaned explants were subjected to primary sterilization by treating the explants with a solution containing Bavistin™ at 1% for 30 minutes and then rinsing with sterile water. The explants were then subjected to secondary sterilization in a laminar flow bench by continuous shaking with mercuric chloride at 0.02% for about 3 minutes and rinsing with sterile distilled water thrice.

### Step 6: Preparation of the Explant and Inoculation in Initiation Medium to Give Multiple Shoots

For final preparation of explants for inoculation, the explant was trimmed without damaging the apical and axillary meristem, taking care to isolate only the meristematic tissue and not other tissues. To avoid the contamination and resultant loss of valuable cultures, each explant was washed and treated separately.

The sterilized explants were inoculated on culture initiation medium consisting of Murashige & Skoog Basal Medium having 3% sucrose and solidified with 0.8% agar, and kept under initial photoperiod of 16 hours under 2000 lux light intensity followed by 8 hours dark period at 25° C. temperature and 60% RH to give multiple shoots.

### Step 7: Transferring the Cultures to Proliferation and Elongation Media

The multiple shoot ratio obtained was around 1:8 with culture media consisting of MS medium augmented with 1.0 mg/L BA and 5 mg/L adenine sulfate. The elongated shoots were subcultured at the same culture conditions as above at a regular interval of about 4 weeks.

### Step 8: Transferring to Rooting Medium

The healthy elongated shoots were transferred to rooting medium consisting of Murashige & Skoog Basal Medium with 5 mg/L IAA and allowed to grow to give well-formed roots thus giving rise to regenerated plants.

### Step: 9: Hardening Protocol for Patchouli in Greenhouse

The regenerated plants were then hardened in a 50-cavity portray having 1″×1″×1.5″ cavity size in a green house with a fan-pad cooling system and fogger to control temperature and relative humidity. The temperature at green house was maintained between 25-30° C. The maximum daylight intensity during hardening was 12000 to 14000 lux. The plants were maintained at a relative humidity of 70-100% for 3-4 weeks initially and finally transferred to polybags with soil and sand 1:1 ratio maintained at 50% shade house.

### Step 10: Transfer of the Hardened Plantlets to the Fields

The plantlets were transferred to the fields by direct sowing or transplanting of the cuttings. The plants propagated by cuttings normally produced seeds within 1 year and the growth was found to be rapid. The success rate in hardening was 90%. All of the plants were healthy and grew well under field conditions.

### SUMMARY OF THE RESULTS

Culture initiation was noticed within 2 weeks after the explants were inoculated in plain MS medium. Bud break was noticed a week after transferring the cultures to light. When initiated cultures were transferred to 1.0 mg/L BA and 5 mg/L adenine sulfate, proliferation began within a month. A two to four fold increase in the number of shoot buds per subculture was noticed up to two subculture cycles and subsequently a four to eight fold increase was observed. After 4 subculture cycles, 90% of regenerated shoots rooted in medium containing 5 mg/L IAA. However, lower concentration of 1 mg/L IAA resulted in only 60% rooting. Ninety percent survival was achieved in green house under controlled conditions. The tissue culture raised plants were successfully transferred to farmers under various agroclimatic conditions and the biomass collected and patchouli oil distilled from the leaves successfully.

Thus, while we have described fundamental novel features of the invention, it will be understood that various omissions and substitutions and changes in the form and details may be possible without departing from the spirit of the invention. For example, it is expressly intended that all combinations of those elements and/or method steps, which perform substantially the same function in substantially the same way to achieve the same results, are within the scope of the invention.

We claim:

1. A method for producing a true-to-type clone of a Pogostemon cablin mother plant comprising
selecting a Pogostemon cablin mother plant;
isolating a meristematic explant from said plant;
culturing said meristematic explant in initiation medium to generate shoots;
culturing said shoots in proliferation and elongation medium to generate elongated shoots, wherein said proliferation and elongation medium comprises benzyl adenine (BA) and adenine sulfate (AS);
culturing said elongated shoots in rooting medium to generate plantlets, wherein said rooting medium comprises indole acetic acid (IAA); and
culturing said plantlets to produce a true-to-type clone of said Pogostemon Cablin mother plant.

2. The method of claim 1, wherein said initiation medium, proliferation and elongation medium, and rooting medium have the same concentration of basal salts.

3. The method of claim 1, wherein said meristematic explant is treated to reduce microbial contamination.

4. The method, of claim 1, wherein said meristematic explant is from a shoot tip or a nodal bud.

5. The method of claim 1, wherein said meristematic explant is from a shoot tip.

6. The method of claim 5, wherein said shoot tip comprises bud tissue.

7. The method of claim 6, wherein said bud tissue is apical bud tissue.

8. The method of claim 7, wherein said bud tissue is axillary bud tissue.

9. The method of claim 1, wherein said BA is at a concentration from about 0.5 mg/L to about 2.0 mg/L.

10. The method of claim 9, wherein said BA is at a concentration selected from the group consisting of: about 0.5 mg/L, about 1.0 mg/L, about 1.5 mg/L, and about 2.00 mg/L.

11. The method of claim 10, wherein said BA is at a concentration of about 0.5 mg/L.

12. The method of claim 1, wherein said AS is at a concentration from about 1 mg/L to about 10 mg/L.

13. The method of claim 12, wherein said AS is at a concentration selected from the group consisting of: about 1.0 mg/L, about 5 mg/L, and about 10 mg/L.

14. The method of claim 13, wherein said AS is at a concentration of about 5 mg/L.

15. The method of claim 1, wherein said IAA is at a concentration from about 1 mg/L to about 10 mg/L.

16. The method of claim 15, wherein said IAA is at a concentration selected from the group consisting of: about 1.0 mg/L, about 5 mg/L, and about 10 mg/L.

17. The method of claim 16, wherein said IAA is at a concentration of about 5 mg/L.

18. The method according to claims hereinabove substantially as herein described with reference to the examples and figures.
